(19) United States
(12) Patent Application Publication
(10) Pub. No.: US 2025/0384975 A1
(43) Pub. Date: Dec. 18, 2025

(54) METHODS AND SYSTEMS FOR OUTPUTTING APHERESIS DATA

(71) Applicant: Terumo BCT, Inc., Lakewood, CO (US)

(72) Inventors: Stephanie St. George, Centennial, CO (US); Justin Rooks, Lakewood, CO (US); Nicole Boticke, Lakewood, CO (US); Daniel Clement, Lakewood, CO (US); Karl Archuleta, Highlands Ranch, CO (US); John Lindner, Morrison, CO (US)

(21) Appl. No.: 19/222,151

(22) Filed: May 29, 2025

Related U.S. Application Data
(60) Provisional application No. 63/659,516, filed on Jun. 13, 2024.

(51) Int. Cl. G16H 10/40

(52) U.S. Cl. CPC G16H 10/40

(57) ABSTRACT

A system for receiving and modifying apheresis data includes at least one memory and at least one processor. The at least one memory is configured to store instructions. The at least one processor is configured to execute the instructions and cause the system to receive data from an apheresis machine following completion of an apheresis procedure, process the data to transform the data into a second format, and save the data in the second format into a database. The data received from the apheresis machine is in a first format and the second format is different from the first format.

### CROSS-REFERENCE TO RELATED APPLICATION

This application claims the benefit of U.S. Provisional Application No. 63/659,516, filed on Jun. 13, 2024. The entire disclosure of the above application is incorporated herein by reference.

### FIELD

The present disclosure relates to systems for receipt and monitoring of apheresis data. The present disclosure also relates to wireless systems for apheresis collection and data processing and output of apheresis data.

### BACKGROUND

This section provides background information related to the present disclosure which is not necessarily prior art.

Therapeutic apheresis systems are designed to collect cells from a donor and to treat a patient. A single machine may be used for both treatment and collection. During collection, whole blood may be collected from a donor, followed by a centrifugal process that separates blood components from the whole blood based on the density of the blood component. During treatment, a patient may be hooked up to the therapeutic apheresis system to receive one or more blood components.

After collection and/or treatment via the therapeutic apheresis system, data from the collection and/or treatment may be recorded and utilized in future reports.

### BRIEF SUMMARY

This section provides a general summary of the disclosure and is not a comprehensive disclosure of its full scope or all of its features.

At least one example embodiment is a system for receiving and modifying apheresis data. The system may include at least one memory and at least one processor. The at least one memory may be configured to store instructions. The at least one processor may be configured to execute the instructions and cause the system to receive data from an apheresis machine following completion of an apheresis procedure, process the data to transform the data into a second format, and save the data in the second format into a database. The data received from the apheresis machine may be in a first format and the second format may be different from the first format.

In at least one example embodiment, the data may be an extensible markup language (XML) data file.

In at least one example embodiment, the processor may be further configured to cause the system to send the data from the database to a customer specified location. In at least one example embodiment, sending the data from the database to the customer specified location may include sending the data in the second format to a second system that is configured to interface with the customer specified location, formatting the data in the second format to a customer specified format, and sending the data in the customer specified format to the customer specified location. In at least one example embodiment, the customer specified format may be at least one of a comma separated value (CSV) file, a text (TXT) file, or an Extensible Markup Language (XML) file. In at least one example embodiment, the data may be an apheresis data file for a patient and the customer specified location may be a patient record for the patient.

In at least one example embodiment, the data received from the apheresis machine may be temporarily stored prior to the data in the second format being saved into the database. In at least one example embodiment, the processor may be further configured to cause the system to delete the data that is temporarily stored after the data in the second format is saved into the database.

In at least one example embodiment, the processor may be further configured to cause the system to request additional data from a third party and receive the additional data from the third party in response to sending a request for the additional data to the third party.

In at least one example embodiment, the processor may be further configured to cause the system to log activity related to the data to store a record of actions taken with respect to the data. In at least one example embodiment, the processor may be further configured to cause the system to store the logged activity for a period of time and delete the logged activity after the period of time. In at least one example embodiment, the period of time may be thirty (30) days.

In at least one example embodiment, processing the data may include verifying a connection to the database and in response to the connection not being verified, repeating the verification of the connection to the database.

In at least one example embodiment, processing the data may include verifying the data and in response to the data being locked or unable to be read, repeating the verification of the data.

Also described herein is a method for receiving and modifying apheresis data. The method may include receiving data from an apheresis machine following completion of an apheresis procedure, processing the data to transform the data into a second format, and saving the data in the second format into a database. The data received from the apheresis machine may be in a first format and the second format may be different from the first format.

In at least one example embodiment, the data may be an extensible markup language (XML) data file.

In at least one example embodiment, the method may further include sending the data from the database to a customer specified location. In at least one example embodiment, sending the data from the database to the customer specified location may include sending the data in the second format to a second system that is configured to interface with the customer specified location, formatting the data in the second format to a customer specified format, and sending the data in the customer specified format to the customer specified location. In at least one example embodiment, the customer specified format may be at least one of a comma separated value (CSV) file, a text (TXT) file, or an Extensible Markup Language (XML) file. In at least one example embodiment, the data may be an apheresis data file for a patient and the customer specified location may be a patient record for the patient.

In at least one example embodiment, the data received from the apheresis machine may be temporarily stored prior to the data in the second format being saved into the database. In at least one example embodiment, the method may further include deleting the data that is temporarily stored after the data in the second format being saved into the database.

In at least one example embodiment, the method may further comprise requesting additional data from a third party and receiving the additional data from the third party in response to sending a request for the additional data to the third party.

In at least one example embodiment, the method may further include logging activity related to the data to store a record of actions taken with respect to the data. In at least one example embodiment, the method may further include storing the logged activity for a period of time and deleting the logged activity after the period of time. In at least one example embodiment, the period of time may be thirty (30) days.

In at least one example embodiment, processing the data may include verifying a connection to the database and in response to the connection not being verified, repeating the verification of the connection to the database.

In at least one example embodiment, processing the data may include verifying the data file and in response to the data being locked or unable to be read, repeating the verification of the data file.

Also described herein is a machine for performing an apheresis procedure. The machine may include a user interface for communicating with a user about the apheresis procedure, a removable panel, and a wireless card. The removable panel may be configured to be removed to access internal components of the machine. The internal components of the machine may include a wireless radio configured to enable wireless connection between the machine and a system remote from the machine.

In at least one example embodiment, the user interface may be configured to enable the user to connect the machine to the system remote from the machine via the wireless connection

In at least one example embodiment, the internal components of the machine may include one or more antennas configured to facilitate the wireless connection between the machine and the system remote from the machine.

Also described herein is a system for storing data from an apheresis procedure. The system may include at least one memory and at least one processor. The at least one memory may be configured to store instructions. The at least one processor may be configured to execute the instructions and cause the system to compile data from the apheresis procedure after completion of the apheresis procedure and generate a machine readable code. The machine readable code may store the data from the apheresis procedure.

In at least one example embodiment, the machine readable code may be a quick response (“QR”) code.

In at least one example embodiment, the machine readable code may be a barcode.

In at least one example embodiment, the processor may be further configured to cause the system to output the machine readable code to a user interface of an apheresis machine.

In at least one example embodiment, the processor may be further configured to cause the system to output the machine readable code to at least one of a printer, a computer external to an apheresis machine, or a database stored on a network accessible by the apheresis machine.

Also described herein is a method for storing data from an apheresis procedure. The method may include compiling data from the apheresis procedure after completion of the apheresis procedure and generating a machine readable code, the machine readable code storing the data from the apheresis procedure.

In at least one example embodiment, the machine readable code may be a quick response (“QR”) code.

In at least one example embodiment, the machine readable code may be a barcode.

In at least one example embodiment, the method may further include outputting the machine readable code to a user interface of an apheresis machine. In at least one example embodiment, the method may further include scanning the machine readable code with a scanner. In at least one example embodiment, the method may further include transferring the data from the apheresis procedure from the scanner to an application after the machine readable code is scanned with the scanner. In at least one example embodiment the scanner may be at least one of a cell phone or a tablet.

In at least one example embodiment, the method may further include outputting the machine readable code to at least one of a printer, a computer external to an apheresis machine, or a database stored on a network accessible by the apheresis machine.

Also described herein is a system for receiving and modifying apheresis data. The system may include at least one memory and at least one processor. The at least one memory may be configured to store instructions. The at least one processor may be configured to execute the instructions and cause the system to receive data from an apheresis machine following completion of an apheresis procedure, process the data to transform the data from a first format to a second format, and save the data in the second format into a database. The second format may be different from the first format.

### BRIEF DESCRIPTION OF THE SEVERAL VIEWS OF THE DRAWINGS

The drawings described herein are for illustrative purposes only of selected embodiments and not all possible implementations and are not intended to limit the scope of the present disclosure.

FIG. 1 is a front view of an apheresis machine according to an example embodiment.

FIG. 2 is a back view of the apheresis machine of FIG. 1 according to an example embodiment.

FIG. 3 is a back view of internal components of the apheresis machine of FIG. 1 according to an example embodiment.

FIG. 4 is a perspective view of an electrical box of the internal components of the apheresis machine of FIG. 1 according to an example embodiment.

FIG. 5 is a perspective view of the apheresis machine of FIG. 1 with a pump cover in an open position according to an example embodiment.

FIG. 6 is a block diagram of a system of the apheresis machine of FIG. 1 suitable for implementing the methods described herein according to an example embodiment.

FIG. 7 is a flow chart of a method for receiving and modifying an end of run file from an apheresis procedure according to an example embodiment.

FIG. 8 is a flow chart of the step 702 of the flow chart of FIG. 7 according to an example embodiment.

FIG. 9 is a flow chart of the step 706 of the flow chart of FIG. 7 according to an example embodiment.

FIG. 10 is a flow chart of a method of sending data from the apheresis machine to a customer specified location according to an example embodiment.

FIG. 11 is a perspective view of the apheresis machine of FIG. 1 displaying a machine readable code according to an example embodiment.

In the appended figures, similar components and/or features may have the same reference label. Further, various components of the same type may be distinguished by following the reference label by a letter that distinguishes among the similar components. If only the first reference label is used in the specification, the description is applicable to any one of the similar components having the same first reference label irrespective of the second reference label.

### DETAILED DESCRIPTION

Example embodiments will now be described more fully with reference to the accompanying drawings.

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected, or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers, and/or sections, these elements, components, regions, layers, and/or sections should not be limited by these terms. These terms may be only used to distinguish one element, component, region, layer or section from another region, layer, or section. Terms such as “first,” “second,” and other numerical terms when used herein do not imply a sequence or order unless clearly indicated by the context. Thus, a first element, component, region, layer, or section discussed below could be termed a second element, component, region, layer, or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

In this application, including the definitions below, the term “module” or the term “controller” may be replaced with the term “circuit.” The term “module” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

The term code, as used above, may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The term memory circuit is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory, tangible computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general-purpose computer to execute one or more particular functions embodied in computer programs. The functional blocks, flowchart components, and other elements described above serve as software specifications, which can be translated into the computer programs by the routine work of a skilled technician or programmer.

The computer programs include processor-executable instructions that are stored on at least one non-transitory, tangible computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (I) descriptive text to be parsed, such as HTML (hypertext markup language), XML (extensible markup language), or JSON (JavaScript Object Notation) (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Swift, Haskell, Go, SQL, R, Lisp, Java®, Fortran, Perl, Pascal, Curl, OCaml, Javascript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

Example embodiments will now be described more fully with reference to the accompanying drawings.

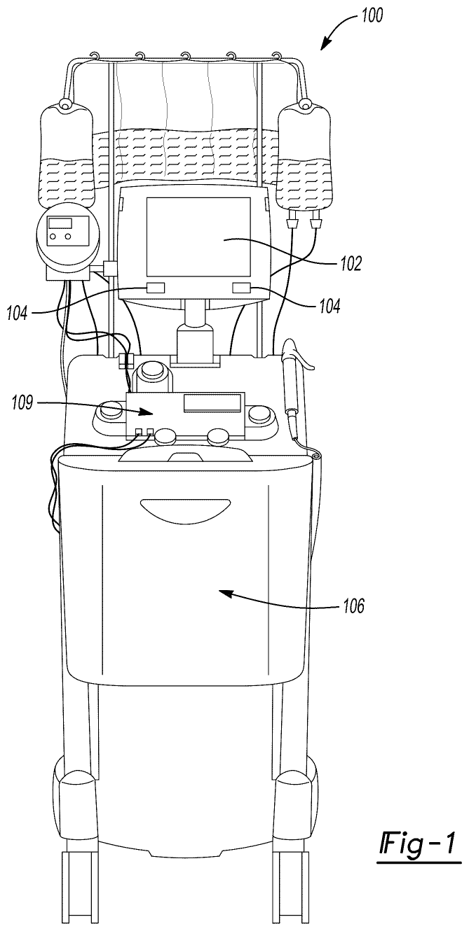

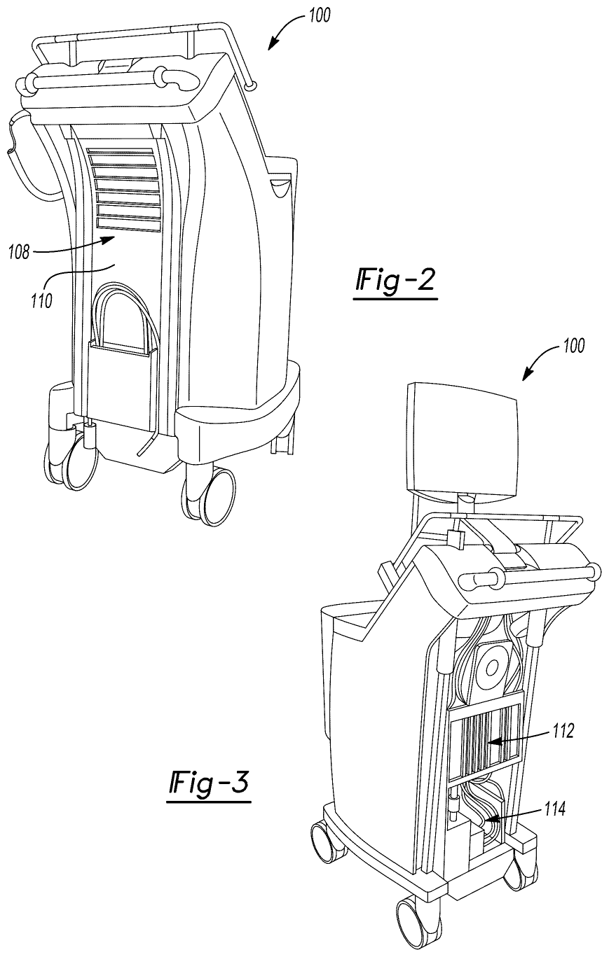

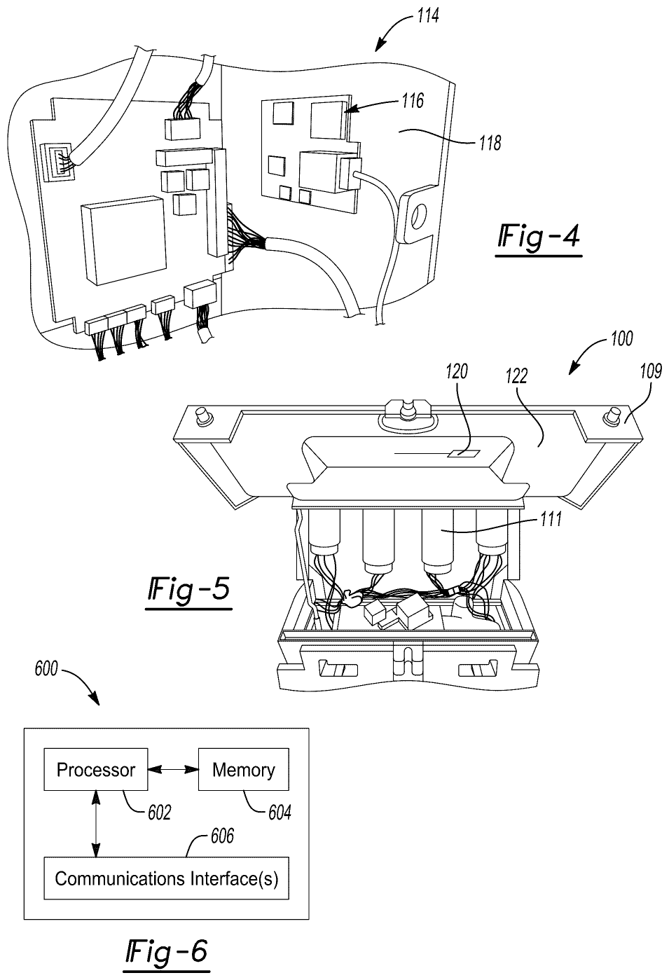

FIGS. 1-5 show an example embodiment of an apheresis machine 100. The apheresis machine 100 may be utilized in apheresis procedures and may have components configured to connect to a patient or user to complete an apheresis procedure. The apheresis machine 100 may additionally include a user interface 102. The user interface 102 may be configured to display information about the patient or user as well as information about the apheresis procedure being completed by the apheresis machine 100. The apheresis machine 100 may additionally include one or more control elements 104. The one or more control elements 104 may be buttons, a keypad, a joystick, or another element capable of allowing a user to interact with the apheresis machine 100.

The apheresis machine 100 may have a first side 106 and a second side 108 opposite the first side 106. The first side 106 may include the one or more control elements 104 and the user interface 102 and thus may be an outward facing or generally front facing side of the apheresis machine 100. The first side 106 may include a pump access panel 109 that may be configured to open to enable access to one or more pumps 111 of the apheresis machine 100. The second side 108 may include a control panel 110 or other access element that may enable access to internal electrical components 112 of the apheresis machine 100.

In at least one example embodiment, the internal electrical components 112 of the apheresis machine 100 may include an electrical box 114. In at least one example embodiment, the electrical box 114 may include a wireless radio such as a wireless card 116. The wireless radio may be referred to herein as the wireless card 116. The wireless card 116 may be mounted on a single board computer (“SBC”) mounting plate 118 of the apheresis machine 100 or on another location of the apheresis machine 100 such that the wireless card 116 is attached to the apheresis machine 100. The wireless card 116 may be configured to enable a wireless connection between the apheresis machine 100 and one or more networks to enable wireless communication between the apheresis machine 100 and one or more network devices connected to the one or more networks. In at least one example embodiment, the wireless card 116 may be powered by either a 3.3V power source or a 5V power source. In at least one example embodiment, the wireless card 116 may be an ethernet to wi-fi bridge. For example, the wireless card may be a Silex IM-100™ ethernet to wi-fi bridge or a Silex SX-590™ ethernet to wi-fi bridge. The wireless card 116 is not limited to the embodiments described herein and may be any wireless card as known in the art.

In at least one example embodiment, the internal electrical components 112 of the apheresis machine 100 may further include at least one circuit card assembly such as a printed circuit board, one or more power sources, and one or more ethernet ports.

The internal components of the apheresis machine 100 may enable integrated wireless capabilities. These capabilities may provide a secure wireless connection between the apheresis machine 100 and a network. This may eliminate the need for a hardwired connection between a network and an apheresis machine 100 which may provide ease of movement of the apheresis machine 100 around a location such as a hospital or donation center. Thus, the wireless capabilities may increase ease of use of the apheresis machine 100.

The apheresis machine 100 may additionally include one or more antennas 120. In at least one example embodiment, the one or more antennas 120 may be located on an inner surface 122 of the pump access panel 109. In at least one example embodiment, the location of the one or more antennas 120 may be configured to avoid metal to prevent blocking of signals received by the one or more antennas 120. The location of the one or more antennas 120 is not limited by the example embodiment disclosed here. The one or more antennas 120 may be located at another location of the apheresis machine 100 to provide connectivity between the apheresis machine 100 and external systems via the wireless card 116 of the apheresis machine 100. In at least one example embodiment, the one or more antennas 120 may include a flex antenna such as EMF2449A1-10MH4L™. Although example embodiments are not limited herein.

In at least one example embodiment, the apheresis machine 100 may also be equipped with Bluetooth® technology. Bluetooth® technology may enable a barcode scanner to be used to transfer information between the apheresis machine 100 and another system such as a smart phone or other external computer. Bluetooth® technology may also enable a wireless connection between the apheresis machine 100 and another system such as a smart phone or other external computer. Both the wireless and Bluetooth® technology capabilities of the apheresis machine 100 may enable improved data transfer and communication between the apheresis machine 100 and external systems.

The apheresis machine 100 may include a system 600. The system 600 may be a computer or other architecture capable of performing the methods and functionality described herein. The system 600 may include at least one processor 602. The at least one processor 602 may be a central processing unit (CPU) or other suitable processor(s). The system 600 may further include a memory 604 such as a random access memory (RAM), read only memory (ROM), or another suitable memory.

The system 600 also may include one or more input/output devices 606. The one or more input/output devices 606 may include a user input device, such as a keyboard, a keypad, a mouse, and the like, a user output device, such as a display, a speaker, and the like, an input port, an output port, a receiver, a transmitter, one or more storage devices, such as a tape drive, a floppy drive, a hard disk drive, a compact disk drive, and the like, as well as various combinations thereof. The methods and processes described herein will be described with respect to the system 600.

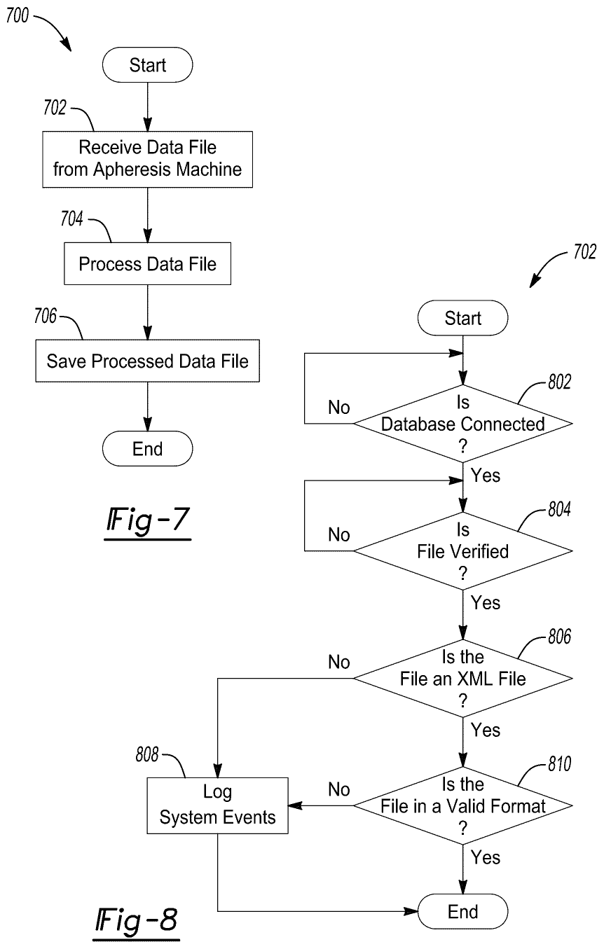

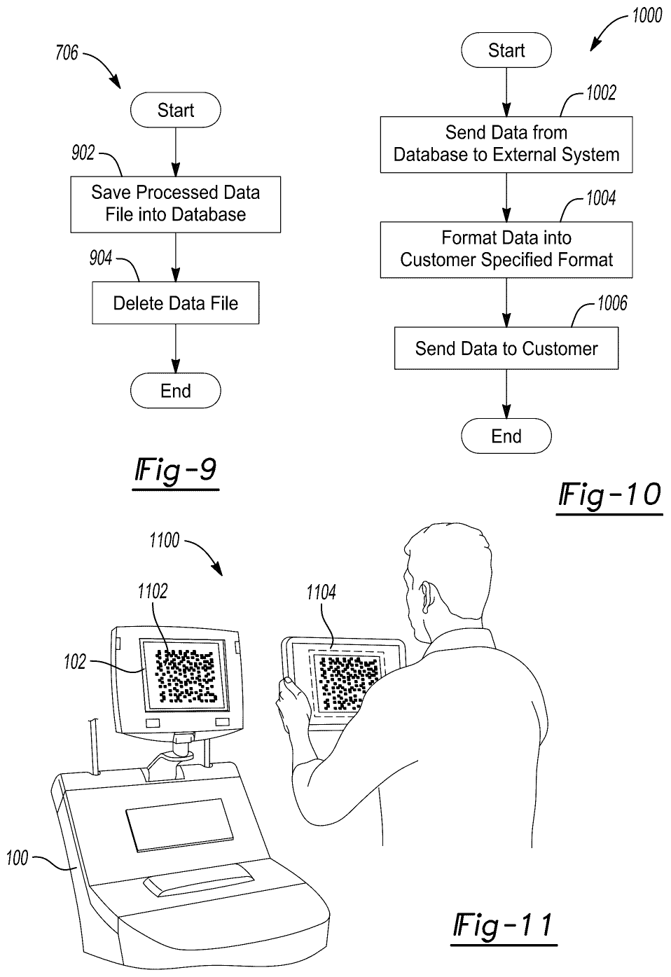

FIG. 7 is a flow chart of a method 700 for receiving and modifying end of run data from an apheresis procedure. Additional details related to the method 700 are further described herein with respect to FIGS. 8 and 9. FIGS. 7-9 are described herein with reference to the processor 602 of FIG. 6 of the apheresis machine 100.

At step 702, the processor 602 receives data from the apheresis machine 100. In at least one example embodiment, the data may be a data file. The data may additionally or alternatively be transferred via an application programming interface (“API”) based system which may transfer the data without it being in a data file. Thus, although described herein with respect to a data file, data that is received and modified after completion of an apheresis procedure is not limited herein. The data file may be an end of run file in at least one example embodiment. The terms data file and end of run file are used interchangeably herein. In particular, when an apheresis procedure is completed an end of run file is generated and stored in a specified folder at a server. In at least one example embodiment, the server may be a customer server that is configured to receive end of run data files that have not yet been processed. In at least one example embodiment, After an apheresis procedure is completed, the apheresis machine 100 may be rebooted and the method 700 begins. Additional details of a method of receiving a data file from the apheresis machine 100 are described below with respect to FIG. 8.

After the end of run file is received, the processor 602 processes the end of run file at step 704. In at least one example embodiment, the processor 602 may map the data in the end of run file to a data entry to process the end of run file. For example, the end of run file may be an XML file that may be converted into a relational structured query language (“SQL”) database. The database may include a combination of discrete fields and payload fields that the data from the end of run file may be mapped to. In at least one example embodiment, the payload fields of the database may store a JavaScript object notation (“JSON”) representation of the data from the end of run data file to allow the data to be dynamic and flexible for future use. In at least one example embodiment, the data from the database may be available external to the database using SQL views that compile the data for consumption and use. Thus, in at least one example embodiment, mapping the data in the end of run file into a data entry may include processing the end of run file to transform the end of run file into a second format where the second format is different from the first format. Processing the end of run file may result in data that is formatted for downstream consumption. In at least one example embodiment, the data may be formatted in at least one of Health Level Seven (“HL7”), Fast Healthcare Interoperability Resources (“FHIR”), TXT, XML, Excel® (“EXC”), or CSV format.

After the end of run file is processed, the processor 602 saves the processed and of run file at step 706. Additional details of a method of saving the processed the end of run file are described below with respect to FIG. 9.

FIG. 8 is a flow chart of the step 702 of the method 700.

In at least one example embodiment, the step 702 may begin at conditional step 802 where a connection to a database is verified by the processor 602. In particular, the processor 602 may be configured to verify whether the apheresis machine 100 is connected to a database. In at least one example embodiment, the database may be the location where the processed data file is saved in step 706.

If the connection is not verified, the processor 602 may retry the connection. In at least one example embodiment, the connection may be retried a specified number of times before the processor 602 shuts down the method 700. In particular, the connection may be checked four times with a two second delay between each connection attempt. In another example embodiment, a different number of connection attempts may be tried with a different time delay between each connection attempt.

If the connection is verified, the processor 602 then verifies the end of run file at conditional step 804. The file verification of conditional step 804 may include checking whether the end of run file is locked or unable to be read by the processor 602. If the end of run file is locked or unable to be read by the processor 602, then the processor 602 may retry the file verification. In at least one example embodiment, the file verification may be retried a specified number of times before the processor 602 shuts down the method 700.

If the end of run file is verified, the processor 602 may determine if the end of run file is an extensible markup language (“XML”) file at conditional step 806. In at least one example embodiment, the file type may be different than an XML file and the processor 602 may determine if the end of run file is a valid type based on a preset file type.

If the end of run file is not an XML file, the processor 602 updates a log with information from the apheresis procedure and the result of the conditional step 806 at step 808. In at least one example embodiment, a log may be kept to record records of activity of the apheresis machine 100. The log may keep records for a specified period of time and may delete the files after the specified period of time. In at least one example embodiment, the specified period of time may be thirty (30) days. In at least one example embodiment, the results of conditional steps 802 and 804 may similarly be logged in the log.

If the end of run file is an XML file, the processor 602 determines if the end of run file is in a valid format at conditional step 810. If the end of run file is not in a valid format, the log is updated with information from the apheresis procedure and the information determined by the processor 602 throughout the method 700. For example, the file type and the file format of the end of run file may be saved in the log.

If the end of run file is in a valid format, the processor 602 processes the end of run file at step 704 as described above with respect to FIG. 7.

FIG. 9 is a flow chart of the step 706 of the method 700.

In at least one example embodiment, the processed end of run file may be the data entry described above with respect to step 704 of FIG. 7. Although described herein as being saved within a database, it should be understood that the data entry may be saved or transferred to another location that may or may not be a database. At step 902, the processor 602 may save the data entry into a database. In at least one example embodiment, the database may have a mix of typed fields and payload fields. The payload fields may be configured to capture the data that differs between protocols that may be executed by the apheresis machine 100. Because there may be different fields per protocol, there may be a large percentage of fields that have null values in the data entry. To create formatting that avoids wide tables with many null values, data of the data entry may be stored in a payload field as Java script object notation (“JSON”). The data entry in the database may be accessed using a structured query language (“SQL”) view.

In at least one example embodiment, the database may be accessible by additional software, programs, databases, and/or other computer systems. For example, the database may be accessible by a third-party software that may be configured to manipulate the data entry to a format that may be utilized by a consumer of the data entry. This may allow the end of run file to be seamlessly integrated with a third party system or data entry without requiring manual input of data. This may improve efficiency and accuracy of recording outputs of apheresis procedures.

In at least one example embodiment, the database may be accessible by a third party system that may import the data entry and transform it into a format that is desirable for use with a medical data record system. For example, the data entry of the database may be transformed into a specified format, configured for export to an end user, and exported in a second specified format to the end user. The second specified format may be different or may be the same as the specified format. For example, the second specified format may be at least one comma separated value (CSV) file, a text (TXT) file, or an Extensible Markup Language (XML) file. The end user may be a medical provider or another user who is importing the data entry derived from the end of run file into a patient record for a patient who underwent the apheresis procedure.

After the data entry has been saved to the database, the processor 602 deletes the end of run file from the specified folder at the server at step 904.

In at least one example embodiment, the processor 602 may additionally be configured for importing data and/or responding to a received message requesting data. For example, the processor 602 may be configured to request data from a third party and receive the requested data from the third party in response to sending the request for data. In at least one example embodiment, the processor 602 may be configured to receive an HL7 message from an electronic medical record (“EMR”) or from an electronic health record (“EHR”). In response to receiving the HL7 message, the processor 602 may be configured to utilize data from an apheresis procedure to respond to the HL7 message.

In at least one example embodiment, the user interface 102 of the apheresis machine 100 may be utilized by a medical professional or another user to initiate and/or monitor the progress of the method 700. Additionally or alternatively, the method 700 may be automatically initiated after completion of an apheresis procedure.

FIG. 10 is a flow chart of a method 1000 of sending data to a customer specified location.

The method 1000 may begin at step 1002 when the processor 602 sends the data entry from the database to a second system that is configured to interface with the customer specified location. In at least one example embodiment, the second system may be a third party or external system. Optionally, the step 1002 may be excluded and the processor 602 and the system 600 may be configured to directly interface with the customer specified location. Although described herein as a customer-specified location, the data may be sent to a downstream location where the data may be processed, analyzed, or used in some manner. In at least one example embodiment, the downstream location may not be a customer-specified location but may be a defined or identified location for receiving the data that may be related or unrelated to a customer.

At step 1004, the processor 602 or the second system may format the data entry to a customer specified format. The customer specified format may be at least one of CSV, TXT, XML, or another format that the data can be formatted into for use by the customer.

At step 1006, the processor 602 or the second system may send the data entry to the customer. In at least one example embodiment, the data entry may be imported into a patient record for a patient who underwent the apheresis procedure.

FIG. 11 is a perspective view 1100 of the apheresis machine 100 displaying a code 1102 on the user interface 102. In at least one example embodiment, after an apheresis process is complete, the code 1102 may be displayed on the user interface 102. The code 1102 may be a quick response (“QR”) code as shown in FIG. 11. In at least one example embodiment, the code may alternatively be a bar code or another code that is machine and/or human readable. The code 1102 may store information about the apheresis procedure. For example, details of the apheresis procedure such as parameters about the apheresis machine 100 and information about a donor or patient that were recorded within the apheresis machine 100 may be encoded within the code 1102.

In at least one example embodiment, a user may use a scanner 1104 to read the code 1102. In at least one example embodiment, the scanner 1104 may be a device capable of reading the code 1102 such as a cell phone, tablet, or other handheld device. In at least one example embodiment, the scanner 1104 may be connected to an application that may input the data from the apheresis procedure. The application may be configured to collect the data stored within the code 1102 and analyze and/or process the data into a format that may be output to a user and/or a medical data record. For example, the data may ultimately be output in a CSV format, an XML format, or as a portable document format (“PDF”).

In at least one example embodiment, the code 1102 may be output by the apheresis machine 100 even when the apheresis machine 100 is not connected to a network. Thus, data from an apheresis procedure may be captured and stored by the code 1102 without the apheresis machine being connected to a network. This may ensure that data is not lost if there is a network connectivity issue and may allow end of run files to be seamlessly stored without requiring manual input of data. If there is network connectivity, the code 1102 may be sent to a designated printer, computer, and/or database to capture the end of run file. This may improve efficiency and accuracy of recording outputs of apheresis procedures.

The systems and methods described herein may ensure that end of run files for apheresis procedures are accurately and efficiently stored. These systems and methods may ensure that patient records are updated to include apheresis procedure results and may not require user input to process the data. Thus, these systems and methods may provide improved apheresis procedures for medical practitioners, patients, and donors.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A system for receiving and modifying apheresis data, the system comprising:
at least one memory configured to store instructions; and
at least one processor configured to execute the instructions and cause the system to
receive data from an apheresis machine following completion of an apheresis procedure, the data being in a first format,
process the data to transform the data into a second format, the second format being different than the first format,
save the data in the second format into a database.

2. The system of claim 1, wherein the data is an extensible markup language (XML) data file.

3. The system of claim 1, wherein the processor is further configured to cause the system to:
send the data from the database to a customer specified location.

4. The system of claim 3, wherein sending the data from the database to the customer specified location comprises:
sending the data in the second format to a second system that is configured to interface with the customer specified location;
formatting the data in the second format to a customer specified format; and
sending the data in the customer specified format to the customer specified location.

5. The system of claim 4, wherein the customer specified format is at least one of a comma separated value (CSV) file, a text (TXT) file, or an Extensible Markup Language (XML) file.

6. The system of claim 3, wherein the data is an apheresis data file for a patient and the customer specified location includes a patient record for the patient.

7. The system of claim 1, wherein the data received from the apheresis machine is temporarily stored prior to the data in the second format being saved into the database.

8. The system of claim 7, wherein the processor is further configured to cause the system to:
delete the data that is temporarily stored after the data in the second format is saved into the database.

9. The system of claim 1, wherein the processor is further configured to cause the system to:
request additional data from a third party; and
receive the additional data from the third party in response to sending a request for the additional data to the third party.

10. The system of claim 1, wherein the processor is further configured to cause the system to:
log activity related to the data to store a record of actions taken with respect to the data;
store the logged activity for a period of time; and
delete the logged activity after the period of time.

11. The system of claim 1, wherein processing the data comprises:
verifying a connection to the database; and
in response to the connection not being verified, repeating the verification of the connection to the database.

12. The system of claim 1, wherein processing the data comprises:
verifying the data; and
in response to the data being locked or unable to be read, repeating the verification of the data.

13. A machine for performing an apheresis procedure, the machine comprising:
a user interface for communicating with a user about the apheresis procedure; and
a removable panel, the removable panel being configured to be removed to access internal components of the machine, the internal components of the machine including a wireless radio configured to enable a wireless connection between the machine and a system remote from the machine.

14. The machine of claim 13, wherein the user interface is configured to enable the user to connect the machine to the system remote from the machine via the wireless connection.

15. The machine of claim 13, wherein the internal components of the machine include one or more antennas configured to facilitate the wireless connection between the machine and the system remote from the machine.

16. A method for storing data from an apheresis procedure, the method comprising:
compiling data from the apheresis procedure after completion of the apheresis procedure; and
generating a machine readable code, the machine readable code storing the data from the apheresis procedure.

17. The method of claim 16, wherein the machine readable code is at least one of a quick response (“QR”) code or a barcode.

18. The method of claim 16, further comprising:
outputting the machine readable code to a user interface of an apheresis machine.

19. The method of claim 18, further comprising:
scanning the machine readable code with a scanner.

20. The method of claim 19, further comprising at least one of:
transferring the data from the apheresis procedure from the scanner to an application after the machine readable code is scanned with the scanner, or
outputting the machine readable code to at least one of a printer, a computer external to the apheresis machine, or a database stored on a network accessible by the apheresis machine.
